(19) United States
(12) Patent Application Publication
(10) Pub. No.: US 2017/0183728 A1
(43) Pub. Date: Jun. 29, 2017

(54) METHODS AND COMPOSITIONS FOR PREDICTING TOBACCO USE

(71) Applicants: Robert PHILIBERT, Iowa City, IA (US); Alexandre TODOROW, St. Louis, MO (US)

(72) Inventors: Robert PHILIBERT, Iowa City, IA (US); Alexandre TODOROW, St. Louis, MO (US)

(21) Appl. No.: 15/301,966

(22) PCT Filed: Apr. 3, 2015
(86) PCT No.: PCT/IB2015/052470

§ 371 Date: Oct. 5, 2016

Related U.S. Application Data
(60) Provisional application No. 61/976,581, filed on Apr. 8, 2014.

(51) Int. Cl. C12Q 1/68; G01N 33/58

(52) U.S. Cl. CPC C12Q 1/6876; G01N 33/58; C12Q 2600/154

(57) ABSTRACT

Provided herein are methods of reliably determining whether or not an individual is a user of tobacco.

### FEDERALLY SPONSORED RESEARCH OR DEVELOPMENT

This invention was made with government support under Grant No. 5R01HD030588-16A1 awarded by National Institutes of Health and Grant No. P30 DA027827 awarded by the National Institute on Drug Abuse. The government has certain rights in the invention.

### TECHNICAL FIELD

This disclosure generally relates to biological methods of determining the smoking status of an individual.

### BACKGROUND

Smoking prevention programs depend on sensitive and valid epidemiological surveillance of the processes surrounding smoking initiation. Currently, many of these analyses are solely dependent on self-report data, which can be inaccurate. Therefore, it is important that the field develop new tools to supplement existing self-reporting procedures and existing biomarkers (e.g., exhaled carbon monoxide levels) during this critical period. A biomarker for smoking that is superior to existing biomarkers could increase the effectiveness of preventive interventions.

### SUMMARY

It is shown that CpG is not merely a proxy for COT, but provides additional information. The derivation of a novel bivariate score is provided herein that uses COT, CpG as well as self-reported data; and it is shown herein that CpG methylation levels are an essential part of the score, above and beyond the information provided by COT levels and the self-reported information.

In one aspect, a method of determining whether or not an individual is a tobacco user is provided. Such a method typically includes the steps of: determining the level of cotinine in a biological sample from the individual; determining the methylation status of at least one CpG dinucleotide in a biological sample from the individual; and correlating the level of cotinine and the methylation status in the biological sample to determine whether or not the individual is a tobacco user. In some embodiments, such a method can further include obtaining self-report data from the individual regarding whether or not the individual is a tobacco user.

In some embodiments, the level of cotinine is determined using ELISA. In some embodiments, the methylation status of the at least one CpG dinucleotide is determined using bi-sulfite treated DNA. In some embodiments, the correlating step comprises applying an algorithm. Representative biological samples include, without limitation, peripheral blood, lymphocytes, urine, saliva, and buccal cells.

In some embodiments, the at least one CpG dinucleotide comprises position 373378 of chromosome 5 in the AHRR gene. Typically, demethylation at position 373378 of chromosome 5 is indicative of previous or current tobacco use. In some embodiments, the at least one CpG dinucleotide comprises position 377358 of chromosome 5 in the AHRR gene or position 399360 of chromosome 5 in the AHRR gene. Typically, demethylation at position 377358 of chromosome 5 or at position 399360 of chromosome 5 is indicative of previous or current tobacco use.

In another aspect, a computer implemented method for determining whether or not an individual is a tobacco user is provided. Such a method typically includes obtaining, at a computer system, information regarding at least one event that is associated with a user; performing one or more predictive calculations for the user, the calculations based, at least in part, on the obtained information; obtaining measured data associated with the user, the measured data comprising one or more measured COT levels and one or more measured CpG methylation status; generating a predictive score based on the obtained information, the predictive calculations, and the measured data; and providing a likelihood of tobacco usage by the user based on the predictive score.

In some embodiments, the information comprises at least one of age, gender, race, ethnicity, tobacco use, and genotype. In some embodiments, the one or more predictive calculations comprises a predicted COT level and/or a predicted CpG methylation status. Generally, the generating a predictive score comprises obtaining a bivariate score between predicted. COT levels and predicted CpG methylation status and measured COT levels and measured CpG methylation status. In some embodiments, the method further includes generating the score using the information and the CpG-methylation status when the predicted COT level for the user and/or the measured COT level for the user is below a threshold. In some embodiments; the method further includes determining the CpG methylation status for the user, wherein a change in methylation status is an indicator of tobacco use.

In one aspect, a computer implemented method for determining whether or not an individual is a tobacco user is provided. Such a method typically includes obtaining self-report data for a user; performing one or more predictive calculations to determine a predicted. COT level, a predicted CpG methylation status and predicted tobacco use of the user; providing a measured COT level and a measured CpG methylation status for the user; generating a predictive score based on the self-report data, the one or more predictive calculations, the measured COT level and the measured CpG methylation status; and outputting a predicted level of tobacco usage based on the predictive score.

In another aspect, a decision support system is provided that includes a processor; a storage device coupled to the processor and storing instructions that, when executed by the processor, cause the processor to perform operations comprising correlating COT levels in an individual and methylation status in the individual with tobacco use by the individual.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a graph showing the cumulative distribution of serum cotinine levels. The distribution makes a sharp transition above 1 ng/dL, with no subjects having values between 1 and 2 ng/dL.

FIG. 2 is a comparison of the methylation levels in DNA from male smokers (n=64) and lifetime male nonsmokers (n=37) at the 146 probes covering the AHRR locus. The average of the nonsmokers is indicated by the red line, whereas the average for smokers, when it diverges from that of the non-smokers, is illustrated by the blue line. The location of the 3 AHRR probes with at least a trend for genome wide significance is illustrated by the double asterisk. The exact ID, methylation values and p-values for the comparisons at each probe are given in Appendix A.

FIG. 3 is a plot showing the relationship between cg05575921 methylation and serum cotinine levels for all 111 subjects. The methylation of cg05575921 is expressed as the non-transformed beta value, which can be roughly viewed as the percent of methylation.

FIG. 4 is a graph showing the relationship between COT levels and daily cigarette consumption (self-reported).

FIG. 5 is a graph showing a simple scatter plot of COT levels vs. CpG methylation.

FIG. 6 is a graph showing only COT levels (COT levels by COT score).

FIG. 7 is a graph showing COT levels in combination with CpG methylation (COT levels by COT/CpG score).

FIG. 8 is a graph showing cluster analysis of COT scores alone.

FIG. 9 is a graph showing cluster analysis of COT scores and CpG methylation.

FIG. 10 is a schematic diagram of an example of a generic computer system 1000.

### DETAILED DESCRIPTION

Methods are described herein that demonstrate increased sensitivity and specificity than existing methods for detecting tobacco use. Specifically, an algorithm that combines features of cotinine levels as well as DNA methylation status at one or more CpG dinucleotides was able to detect or predict tobacco use with a much higher success rate than that of either method alone.

### Cotinine and Measuring Cotinine Levels

Cotinine, (5S)-1-methyl-5-(3-pyridyl)pyrrolidin-2-one, is an alkaloid found in tobacco and is a metabolite of nicotine.

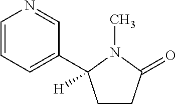

Cotinine has an in vivo half-life of approximately 20 hours, and is typically detectable for several days (e.g., 4, 5, 6 or 7 days, e.g., up to one week) after the use of tobacco. Cotinine can be detected in a number of biological samples including, without limitation, blood, urine, and saliva, although it would be appreciated by a skilled artisan that cotinine concentrations in urine average four-fold to six-fold higher than those in blood or saliva (Avila-Tang et al., 2011, Tobacco Control, 2011-050298), typically making urine a more sensitive biological sample from which low-concentration exposure can be detected.

Cotinine assays provide a quantitative measurement of tobacco use and also permits the measurement of exposure to second-hand smoke (e.g., passive smoking) (Florescu et al., 2009, Therapeutic Drug Monitor, 31(1):14-30. Simply by way of example, when the biological sample is blood, cotinine levels <10 ng/mL are considered to be consistent with no active smoking; values of 10 ng/mL to 100 ng/mL are associated with light smoking or moderate passive exposure; and levels above 300 ng/mL are seen in heavy smokers (e.g., more than 20 cigarettes a day). Simply by way of example, when the biological sample is urine, values between 11 ng/mL and 30 ng/mL are associated with light smoking or passive exposure; and levels in active smokers typically reach 500 ng/mL or more.

Although the above-indicated numbers are used in the art as general guidelines, significant variability is still observed. For example, users of menthol tobacco can retain cotinine in the blood for a longer period of time because menthol can compete with the enzymatic metabolism of cotinine (Ham, 2002, Center for the Advancement of Health, Science Blog). In addition, males generally have higher plasma cotinine levels than females (Gan et al., 2008, Nicotine & Tobacco Res., 10(8):1293-300), and African-Americans generally have higher plasma cotinine levels than Caucasians (Wagenknecht et al., 1990, Am. J. Public Health, 80(9): 1053-6).

In addition, plasma cotinine levels at steady state are determined by the amount of cotinine formation and the rate of cotinine removal, both of Which are mediated by a P450 enzyme, CYP2A6 (Zhu et al., 2013, Cancer Epidem., Biomarkers & Prevention, 22(4):708-18). CYP2A6 activity has been shown to differ by gender (estrogen induces CYP2A6) and race (due to genetic variation). Therefore, cotinine has been shown to accumulate in individuals with slower CYP2A6 activity, which can result in substantial differences in cotinine levels between different individuals that use the same or essentially the same amount of tobacco.

Based on the above, and as explained in more detail below, the presence and/or level of cotinine in a biological sample is not a definitive or conclusive indication of tobacco use.

### Methylation of Nucleic Acids and Determining the Methylation Status of Nucleic Acids

CpG islands are stretches of DNA in which the frequency of the CpG sequence is higher than other regions. The “p” in the term CpG designates the phosphodiester bond that binds the cysteine (“C”) nucleotide and the guanine (“G”) nucleotide. CpG islands are often located around promoters and are often involved in regulating the expression of a gene (e.g., housekeeping genes). Generally, CpG islands are not methylated when a sequence is expressed, and methylated to suppress expression (or “inactivate” the gene).

The methylation status of one or more CpG dinucleotides in genomic DNA or in a particular nucleic acid sequence can be determined using any number of biological samples, such as blood, urine, saliva, or buccal cells. In certain embodiments, a particular cell type, e.g., lymphocytes, basophils, or monocytes, can be obtained (e.g., from a blood sample) and the DNA evaluated for its methylation status.

The methylation status of genomic DNA, of a CpG-island, or of one or more specific CpG dinucleotides can be determined by the skilled artisan using any number of methods. The most common method for evaluating the methylation status of DNA begins with a bisulfite-based reaction on the DNA (see, for example, Frommer et al., 1992, PNAS USA, 89(5):1827-31). Commercial kits are available for bisulfite-modifying DNA. See, for example, EpiTect Bisulfite or EpiTect Plus Bisulfite Kits (Qiagen).

Following bisulfite modification, the nucleic acid can be amplified. Since treating DNA with bisulfite deaminates unmethylated cytosine nucleotides to uracil, and since uracil pairs with adenosine, thymidines are incorporated into DNA strands in positions of unmethylated cytosine nucleotides during subsequent PCR amplifications.

In some embodiments, the methylation status of DNA can be determined using one or more nucleic acid-based methods. For example, an amplification product of bisulfite-treated DNA can be cloned and directly sequenced using recombinant molecular biology techniques routine in the art. Software programs are available to assist in determining the original sequence, which includes the methylation status of one or more nucleotides, of a bisulfite-treated. DNA (e.g., CpG Viewer (Carr et al., 2007, Nucl. Acids Res., 35:e79)). Also for example, amplification products of bisulfite-treated. DNA can be hybridized with one or more oligonucleotides that, for example, are specific for the methylated, bisulfite-treated DNA sequence, or specific for the unmethylated, bisulfite-treated DNA sequence.

In some embodiments, the methylation status of DNA can be determined using a non-nucleic acid-based method. A representative non-nucleic acid-based method relies upon sequence-specific cleavage of bisulfite-treated DNA followed by mass spectrometry (e.g., MALDI-TOF MS) to determine the methylation ratio (methyl CpG/total CpG) (see, for example, Ehrich et al., 2005, PNAS USA, 102:15785-90), Such a method is commercially available (e.g., MassARRAY Quantitative Methylation Analysis (Sequenom, San Diego, Calif.)).

Methylated Nucleic Acid Sequences Associated with Tobacco Use

A number of CpG dinucleotides have been shown to be methylated, demethylated, or hypermethylated in individuals that use tobacco (relative to non-users). For example, the methylation status of CpG dinucleotides within the sequence encoding the aryl hydrocarbon receptor repressor (AHRR), also known as aryl-hydrocarbon hydroxylase regulator (AHHR), or monoamine oxidase A (MAOA) have been associated with tobacco use (e.g., prior tobacco use, current tobacco use). AHRR is a feedback inhibition modulator of the aryl hydrocarbon receptor (AhR) signaling cascade, while MAOA is an enzyme that deaminates norepinephrine, epinephrine, serotonin, and dopamine.

The methylation status (e.g., changes in the methylation status) of one or more CpG islands and/or particular CpG dinucleotides correlated with tobacco use have been described in the literature. See, for example, U.S. Pat. No. 8,637,652; and Dogan et al. (2014, BMC Genomics, 15:151); Philibert et al. (2013, Clin. Epigenetics, 5:19); Philibert et al. (2012; Epigenetics, 7:1331-8); Philibert et al. (2012, J. Leukoc. Biol., 92:621-31); Monick et al. (2012, Am. J. Med. Genet. B. Neuropsychiatr. Genet., 159B:141-51); Philibert et al. (2010, Am. J. Med. Genet. B. Neuropsychiatr. Genet., 153B:619-28); and Philibert et al. (2008, Am. J. Med. Genet. B. Neuropsychiatr. Genet., 147B:565-70); each of which are incorporated herein by reference in its entirety.

For example, the methylation status of certain CpG dinucleotides within the AHRR sequence has been correlated with tobacco use (e.g., demethylation at position 373378 of chromosome 5; demethylation at position 377358 of chromosome 5; demethylation at position 399360 of chromosome 5). The methylation status of additional nucleotides within the AHRR sequence in smokers is shown in Appendix A and also in U.S. Pat. No. 8,637,652. In addition, the methylation status of certain CpG dinucleotides within the MAOA sequence has been correlated with tobacco use (e.g., demethylation in the first and second CpG islands in the promoter of the monoamine oxidase A (MAOA) sequence (e.g., from about −45 CpG residues to about +15 CpG residues from the CpG at the transcription start site (TSS))). Further, Appendix B shows the methylation status of over 900 loci, including AHRR and MAOA sequences, each of which demonstrates a significant association with tobacco use (Dogan et al., 2014, BMC Genomics, 15:151).

Any of the CpG dinucleotides in which methylation status has been associated with tobacco use can be used in the methods herein to increase the predictive value. In addition, it would be appreciated that the methylation status of one or more neighboring CpG dinucleotides can be in linkage disequilibrium with the methylation status of a CpG dinucleotide having significance with tobacco use (see, for example, Philibert et al., 2009, Am. J. Med. Genet. B. Neuropsychiatr. Genet., 153B:619-28) and, therefore, the methylation status of those neighboring CpG dinucleotides can be used in the methods described herein. Further, it would be appreciated that the greater the changes are in the methylation status, the greater the tobacco use. See, for example, Philibert et al., 2012, Epigenetics, 7:1-8.

As used herein, nucleic acids can include DNA and RNA, and includes nucleic acids that contain one or more nucleotide analogs or backbone modifications. A nucleic acid can be single stranded or double stranded, which usually depends upon its intended use.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

A vector containing a nucleic acid (e.g., a nucleic acid that encodes a polypeptide) also is provided. Vectors, including expression vectors, are commercially available or can be produced by recombinant DNA techniques routine in the art. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6×His tag, glutathione S-transferase (GST))

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid (e.g., in-frame). Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Vectors as described herein can be introduced into a host cell. As used herein, “host cell” refers to the particular cell into which the nucleic acid is introduced and also includes the progeny or potential progeny of such a cell. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

Oligonucleotides for amplification or hybridization can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 (e.g., 16, 18, 20, 21, 22, 23, 24, or 25) nucleotides in length. Designing oligonucleotides to be used as hybridization probes can be performed in a manner similar to the design of amplification primers. In some embodiments, hybridization probes can be designed to distinguish between to targets that contain different sequences (e.g., a polymorphism or mutation, e.g., the methylated vs. non-methylated sequence in the bisulfite-treated DNA).

Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tin between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. Simply by way of example, high stringency conditions typically include a wash of the membranes in 0.2×SSC at 65° C.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, Calif.).

A nucleic acid sequence, or a polypeptide sequence, can be compared to one or more related nucleic acid sequences or polypeptide sequences, respectively, using percent sequence identity. In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31(13):3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gin, Glu, Arg, and Lys; and residue-specific gap penalties: on. ClustalW can be run, for example, at the Baylor College of Medicine Search Launcher website or at the European Bioinformatics Institute website on the World Wide Web.

Changes can be introduced into nucleic acid coding sequences using, for example, mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor. N.Y.; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid. Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

### Algorithm and Digital Methods of Implementing the Algorithm

Various implementations of the systems and techniques described herein can be realized in digital electronic circuitry, integrated circuitry, computer hardware, firmware, software, and/or combinations thereof. These various implementations can include implementation in one or more computer programs that are executable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. To provide for interaction with a user, the systems and techniques described herein can be implemented on a computer having a display device for displaying information to the user and a keyboard and a pointing device by which the user can provide input to the computer. Other kinds of devices can be used to provide for interaction with a user as well; for example, feedback provided to the user can be any form of sensory feedback (e.g., visual feedback; auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication.

For example, a computer implemented method is provided that can be used to determine whether or not an individual is a tobacco user. As a first step, information can be obtained regarding at least one event that is associated with a user or a plurality of users. As used herein, events refer to various demographic information (e.g., age, gender, race, ethnicity, genotype) as well as self-reported tobacco use (e.g., daily, weekly, etc.).

Next, one or more calculations can be performed to determine (e.g., predict) a COT level (e.g., a predicted COT level) and a CpG methylation status (e.g., a predicted CpG methylation status) for the user or the plurality of users. As described herein, the calculations are based, at least in part, on the information obtained from the user or the plurality of users regarding one or more events.

In addition to predicting a COT level and a CpG methylation status for the user or the plurality of users, actual COT levels (e.g., measured COT levels) and at least one actual CpG methylation status (e.g., measured CpG methylation status) can be obtained for the user or the plurality of users. Methods of obtaining measured COT levels and at least one measured CpG methylation status are known in the art and are described herein.

Based on the information obtained from the user or the plurality of users regarding one or more events, the predicted COT levels and CpG methylation status, and the measured COT levels and CpG methylation status, a score (e.g., a bivariate score) is generated and can be produced as an output. The score is indicative of tobacco use by the user or plurality of users.

In some embodiments, the predicted COT level and/or the measured COT level for the user or plurality of users is below a certain threshold. In such instances, a score can be generated using the information regarding the one or more events and the CpG methylation status.

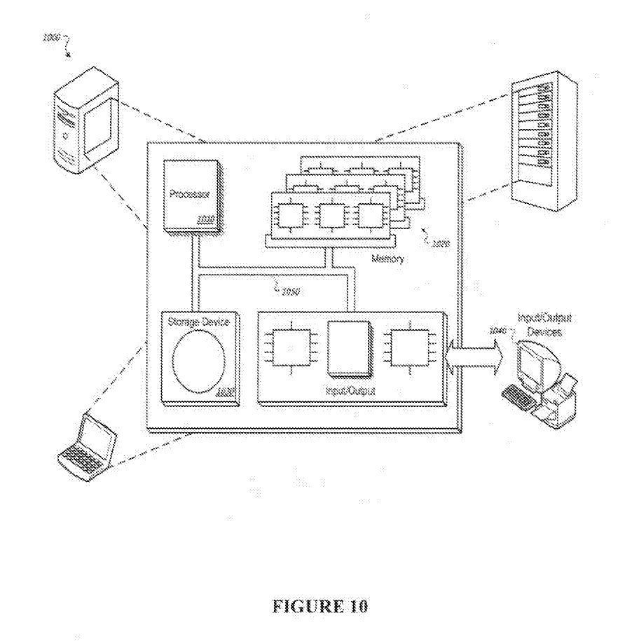

FIG. 10 is a schematic diagram of an example of a generic computer system 1000. In some implementations, the system 1000 can be used for the operations described above.

The system 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the system 1000. In one implementation, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the system 1000. In one implementation, the memory 1020 is a computer-readable medium. In one implementation, the memory 1020 is a volatile memory unit. In another implementation, the memory 1020 is a non-volatile memory unit.

The storage device 1030 is capable of providing mass storage for the system 1000. In one implementation, the storage device 1030 is a computer-readable medium. In various different implementations, the storage device 1030 may be a floppy disk device, a hard disk device, an optical disk device, or a tape device.

The input/output device 1040 provides input/output operations for the system 1000. In one implementation, the input/output device 1040 includes a keyboard and/or pointing device. In another implementation, the input/output device 1040 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### Examples

### Example 1—Subjects

The 107 subjects featured in these analyses are drawn from the Adults in the Making (AIM) project which is a longitudinal study of young African Americans as they transition from adolescence into early adulthood (Brody et al., 2012, J. Consult. Clin. Psychol., 80:17-28). Youths were enrolled in the study when they were 16 years of age. At Wave 1, among youths' families, median household gross monthly income was below $2,100 and mean monthly per capita gross income was below $900.

Families were contacted and enrolled by community liaisons residing in the counties where the participants lived. The community liaisons were African American community members who worked with the researchers on participant recruitment and retention. At all data collection points, parents gave written consent to minor youths' participation, and youths gave written assent or consent to their own participation. To enhance rapport and cultural understanding, African American university students and community members served as field researchers to collect data. At the home visit, self-report questionnaires were administered privately via audio computer-assisted self-interviewing technology on a laptop computer. Youth were compensated for their participation with $50 after each assessment. All protocols and procedures used in the AIM project were approved by the University of Georgia Institutional Review Board.

As a part of the self-report assessment, at each wave of data collection, the subjects were asked “In the past month, how often did you smoke cigarettes?” The number of cigarettes given in reply was used as that year's estimated average monthly consumption with that number being divided by 20 to give the number of packs smoked. A positive response at any time point from a subject resulted in the categorization of that subject as a smoker for the given wave.

### Example 2—Procedures

Approximately 6 months after the collection of the Wave 4 data, the subjects were phlebotomized to provide sera and DNA for the proposed studies. Their average age was 22. The DNA for the current studies was prepared from lymphocyte (mononuclear) cell pellets as previously described (Philibert et al., 2012, Epigenetics, 7). Sera were prepared using serum separator tubes and were frozen at −80° C. after preparation until use.

Genome wide DNA methylation was assessed using the illumina (San Diego, Calif.) HumanMethylation450 Beadchip by the University of Minnesota Genome Center (Minneapolis, Minn.) using the protocol specified by the manufacturer as previously described (Monick et al., 2012, Am. J. Med. Genet., Part B Neuropsychiatric Genet., 159:141-51). This chip contains 485,577 probes recognizing at least 20216 transcripts, potential transcripts or CpG islands (from the Genome Reference Consortium human genome build 37 (GRCh37)). Subjects were randomly assigned to 12 sample “slides” with groups of 8 slides representing the samples from a single 96 well plate being bisulfite converted in a single batch. Four replicates of the same DNA sample were also included to monitor for slide to slide and batch bisulfite conversion variability with the average correlation co-efficient between the replicate samples being 0.997. The resulting data were inspected for complete bisulfite conversion and average beta values for each targeted CpG residue determined using the Illumina Genome Studio Methylation Module, Version 3.2, The resulting data were then cleaned using a PERL based algorithm to remove those beta values whose detection p-values, an index of the likelihood that the observed sequence represents random noise, were greater than 0.05.

Genome wide linear regression analyses of the log transformed data were conducted using MethLAB, version 1.5. using a previously described procedures (Philibert et al., 2012, Epigenetics, Kilaru et al., 2012, Epigenetics, 7:225-9), All the analyses were controlled for both batch and slide. Correction for multiple comparisons was accomplished by using the False Discovery Rate method using an alpha of 0.05 and a subroutine within MethLAB (Benjamin et al., 1995, J. Royal Statist. Soc., Series B, Methodol., 57:289-300. As noted in the results, the regression analyses which were controlled for batch and slide contrasted the log transformed beta values of those who denied ever smoking and had serum cotinine levels <1.0 ng/dL (n=37) to those with serum cotinine levels >2.0 ng/dL (n=64).

### Example 3—Statistical Analysis

The analyses of clinical, serological and single point methylation data were analyzed using the suite of general linear model algorithms contained in JMP, version 10 (SAS Institute, Cary, USA) as indicated in the text.

### Example 4—Results

The clinical and demographic characteristics of the 107 AIM subjects who participated in the study are given in Table 1. The subjects averaged 22 years of age. Nearly 54% of the subjects reported smoking at least one prior cigarette during our clinical interviews. The amount of self-reported smoking tended to be rather light, with the 35 subjects who reported smoking at the last wave of data reporting an average daily consumption of 8±7 cigarettes.

TABLE 1
Clinical and Demographic Characteristics of the Subjects

| | |
|---|---|
| N | 107 |
| Age | 22.0 ± 1.3 |

Smoking Status (self-reporting)

| | |
|---|---|
| Never | 49 |
| Wave 1-3 only | 23 |
| Wave 4 | 35 |
| Average cigarette consumption in Wave 4 smokers | 8 ± 7/day |

Pack year history in Wave 4 smokers

| | |
|---|---|
| ≦1 pack year | 24 |
| 1-2 pack years | 5 |
| >2 pack years | 6 |

Serum Cotinine Levels (ng/ml)

| | |
|---|---|
| <1.0 | 43 |
| 1 < x < 2.0 | 0 |
| >2.0 | 64 |
| Average Cotinine level in those with serum cotinine | 80 ± 58 |
| levels >2 ng/ml | |

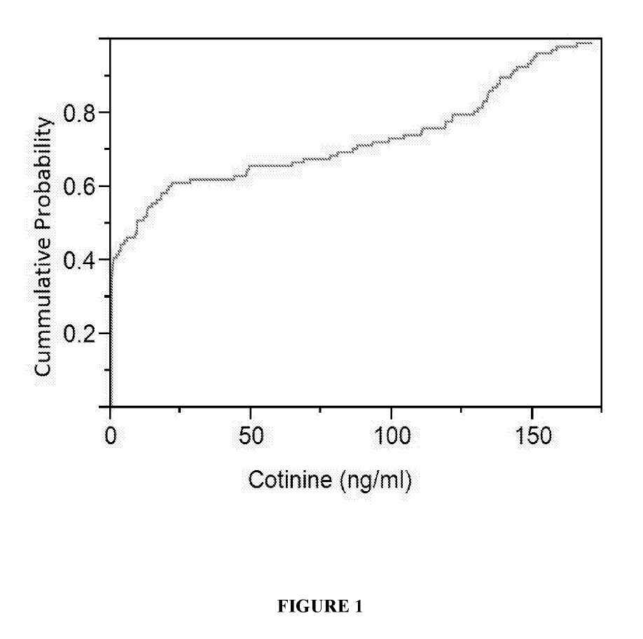

Because the DNA samples were collected approximately 6 months after the collection of Wave 4 data and self-report, data may often be an under report of actual smoking consumption (Kandel et al., 2006, Nic. Tob, Res., 8:525-37; Caraballo et al., 2004, Nic. Tob. Res., 6:19-25), serum cotinine levels of each of the subjects were examined. FIG. 1 illustrates the cumulative frequency distribution of the serum cotinine levels. As the figure illustrates, there was a sharp dog leg break in the distribution of values with 44 (41%) of the subjects having levels of <1 ng/ml, no subjects having values between 1 and 2 ng/dl and 64 (59%) of the subjects having serum cotinine levels of >2 ng/dl (designated hereafter as positive cotinine values). Of considerable interest, 23 of the 64 subjects who denied smoking at all four waves, including the last interview conducted 6 months prior to the blood draw, had serum cotinine levels of >2.0 ng/dL.

As the first step of the main epigenetic analyses, genome wide analysis of the relationship of smoking to DNA methylation was conducted. Because the above serum cotinine data suggest that self-reported smoking status may not be reliable, serum cotinine levels were chosen as the indicator of current smoking status. The DNA methylation status of those 64 subjects was contrasted with serum cotinine levels >2 ng/ml with those 37 subjects who consistently denied smoking through all four waves of data collection and who had negligible levels of serum cotinine (<1.0 ng/ml). Because the previous work at monoamine oxidase A (MAW) showed that smoking cessation is associated with a highly variable remodeling of the MAOA DNA methylation signature, the data from the 6 subjects with serum cotinine levels <1.0 ng/dL but with positive self-reported history of smoking were not included in the genome wide contrasts (Philibert et al., 2010, Am. Med. Genet., 153B:619-28).

Table 2 lists the 30 most significant findings with respect to the data from those 98 subjects. Consistent with prior studies, cg05575921 was the probe most highly associated with smoking status with a False Discovery Rate (FDR) corrected p-value of p<0.002 (Non-smoker (NS) greater than Smokers (S); NS mean 0.85, S mean 0.74, 95% confidence interval 0.82 to 0.87, and 0.72 to 0.76, respectively). A second probe from AHRR, cg21161138, also attained genome wide significance with a FDR corrected p-value of p<0.03 (NS greater than S; NS mean 0.73, S mean 0.69, 95% confidence interval 0.72 to 0.75, and 0.68 to 0.70, respectively). Finally, there was a trend for association at third AHRR probe locus, cg26703534 (NS greater than S; NS mean 0.69, S mean 0.64, 95% confidence interval 0.68 to 0.70, and 0.63 to 0.65; respectively). Methylation at MYO1G probe cg22132788, which was reported to be differentially methylated in DNA prepared from newborns of smoking mothers (Joubert et al., 2012, Environ. Health Perspect., 120:doi:10.1289/ehp.trp083112), was the fourth-ranked probe with a genome wide corrected p-value of p<0.144.

TABLE 2
The 30 most significantly associated probes in DNA from male subjects

| | | | | Average Beta | | | |
|---|---|---|---|---|---|---|---|
| | | | | Values | | | |
| | | | Island | | Non- | | Corrected |
| Probe ID | Gene | Place | Status | Smoker | smoker | T-test | P-value |
| cg05575921 | AHRR | Body | N | 0.74 | 0.85 | 4.92E−09 | 0.002 |
| | | | Shore | | | | |
| cg21161138 | AHRR | Body | | 0.69 | 0.73 | 1.18E−07 | 0.029 |
| cg26703534 | AHRR | Body | S Shelf | 0.64 | 0.69 | 4.72E−07 | 0.076 |
| cg22132788 | MYO1G | Body | Island | 0.94 | 0.88 | 1.19E−06 | 0.144 |
| cg17072268 | PLD3 | TSS1500 | N | 0.82 | 0.84 | 1.11E−05 | 0.999 |
| | | | Shore | | | | |
| cg12108912 | TMEM177 | TSS1500 | N | 0.79 | 0.80 | 1.33E−05 | 0.999 |
| | | | Shore | | | | |
| cg12803068 | MYO1G | Body | S Shore | 0.83 | 0.76 | 1.61E−05 | 0.999 |
| cg22904815 | | | N | 0.44 | 0.48 | 1.65E−05 | 0.999 |
| | | | Shore | | | | |
| cg25628057 | ATAD3B | Body | S Shore | 0.87 | 0.88 | 3.04E−05 | 0.999 |
| cg04521543 | TMEM18 | 3′UTR | | 0.83 | 0.82 | 3.29E−05 | 0.999 |
| cg11270237 | | | N | 0.34 | 0.36 | 3.61E−05 | 0.999 |
| | | | Shore | | | | |
| cg00498653 | | | Island | 0.15 | 0.17 | 3.80E−05 | 0.999 |
| cg22537081 | TBRG4 | TSS200 | Island | 0.03 | 0.03 | 3.94E−05 | 0.999 |
| cg23311108 | | | | 0.33 | 0.36 | 5.29E−05 | 0.999 |
| cg27312872 | C1orf212 | Body | N Shelf | 0.83 | 0.84 | 5.50E−05 | 0.999 |
| cg13960339 | ZIM2 | TSS200 | Island | 0.51 | 0.53 | 6.27E−05 | 0.999 |
| cg07918390 | GPSM3 | TSS1500 | Island | 0.04 | 0.04 | 6.60E−05 | 0.999 |
| cg16148833 | | | | 0.72 | 0.74 | 6.85E−05 | 0.999 |
| cg27072683 | NDUFB8 | TSS1500 | S Shore | 0.25 | 0.27 | 7.28E−05 | 0.999 |
| cg16579844 | RNASE4 | 1stExon | S Shore | 0.04 | 0.04 | 7.54E−05 | 0.999 |
| cg08939942 | | | | 0.91 | 0.92 | 7.59E−05 | 0.999 |
| cg25202390 | MRPL30 | 1stExon | Island | 0.18 | 0.16 | 9.23E−05 | 0.999 |
| cg04097463 | | | S Shelf | 0.84 | 0.86 | 9.76E−05 | 0.999 |
| cg19192585 | | | Island | 0.03 | 0.03 | 9.78E−05 | 0.999 |
| cg18075691 | | | N Shelf | 0.41 | 0.36 | 9.87E−05 | 0.999 |
| cg20215007 | ZNF467 | 5′UTR | N | 0.19 | 0.21 | 0.0002 | 0.999 |
| | | | Shore | | | | |
| cg11467141 | | | Island | 0.95 | 0.92 | 0.0002 | 0.999 |
| cg00534919 | C1orf26 | Body | | 0.10 | 0.10 | 0.000113816 | 0.999 |
| cg08771171 | CTNNA1 | Body | | 0.80 | 0.82 | 0.0001182 | 0.999 |
| cg21029030 | MIF4GD | TSS1500 | Island | 0.02 | 0.02 | 0.000118582 | 0.999 |

All average methylation values are non-log transformed beta-values.
Island status refers to the position of the probe relative to the island.
Classes include: 1) Island, 2) N (north) shore, 3) S (south) shore, 4) N (north shelf), 5) S (south) shelf and 6) blank denoting that the probe does not map to an island.

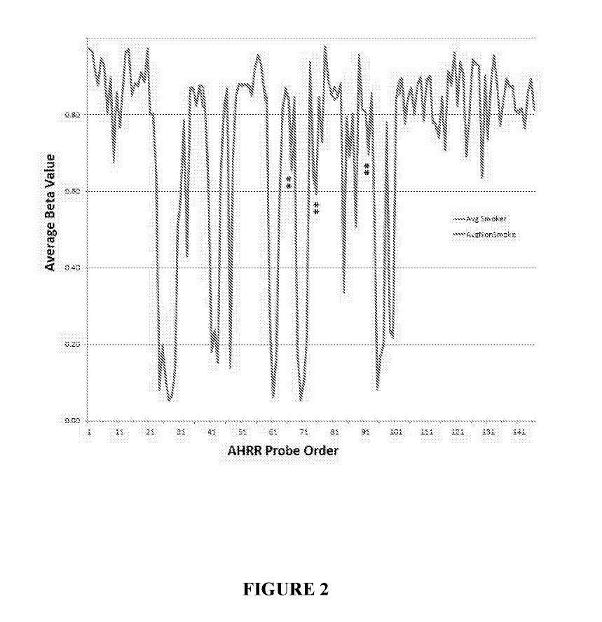

Because AHRR is a complexly regulated gene (e.g., at least 5 CpG islands) with 146 probes mapping to it, the relationship of smoking status to methylation at each these 146 probes was examined. FIG. 2 illustrates the degree of methylation at each of those residues in the smokers and nonsmokers, while Table 3 gives the ID, position, sequence exact averages and p-values obtained for each probe. As FIG. 2 and Table 3 together demonstrate, 10 probes clustering to 4 discrete areas have nominal significance values of <1×10⁻³. Notably, at all 10 of these AHRR probes with a nominal significance value of <1×10⁻³, smoking was associated with demethylation.

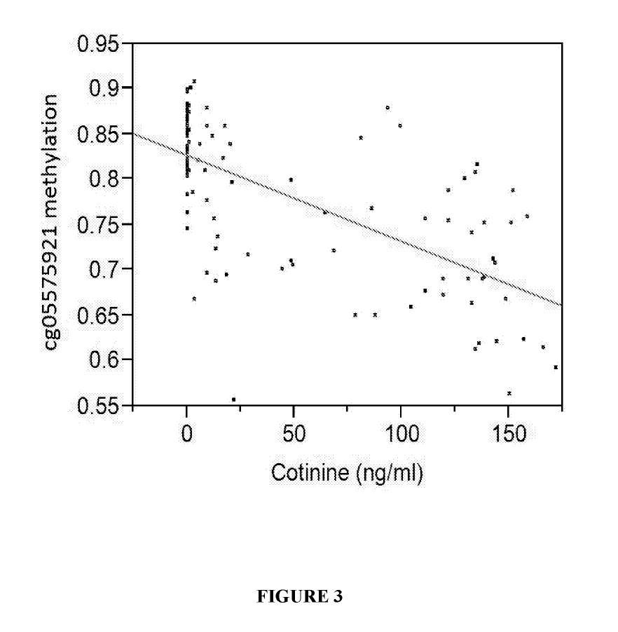

Because methylation at cg05575921 was once again the most highly associated residue in terms of DNA methylation, the relationship between methylation status at that residue and serum cotinine levels was analyzed. Using the data from all 107 subjects, it was found that methylation status using probe cg05575921 (corresponding to position 373378 of chromosome 5) was highly correlated with serum cotinine levels (FIG. 3, adjusted R²=0.42, p<0.0001). Methylation status at the other two highly associated AHRR residues, detected using probe cg26703534 (corresponding to position 377358 of chromosome 5; adjusted R²=0.28, p<0.0001) and cg21161138 (corresponding to position 399360 of chromosome 5; adjusted R²=0.19, p<0.0001), was also highly correlated although the proportion of the variance explained was considerably less.

### Example 5—Cotinine Levels and Reported Cigarette Consumption

The data were collected from 106 males and 307 females, 99 of whom report being current smokers (median number of cigarettes smoked daily: 10). As expected, individuals who report smoking at least one cigarette daily present with significantly higher COT levels (median COT: 159.4 ng/ml, IQR: 167.5-148.5) compared to non-regular smokers (median COT: 0.01, IQR: 0.00-0.63; p<0.0001, Wilcoxon test). Using COT levels alone, the optimum classifier for individuals who report reaches a sensitivity of 86% and a specificity of 89%, which results in a positive predictive value (PPV) of 79% and a negative predictive value (NPV) of 93%. Overall, 88% of individuals are correctly classified using COT levels alone, and the AUC=0.92 (95% CI=0.89-0.95). These predictive values, which are based on self-report data, are slightly lower, but not meaningfully so, than those reported in the well-controlled (confirmed smokers/non-smokers) studies, such as that of Benowitz et al. (2009, Am. J. Epidemiol., 169:236-48).

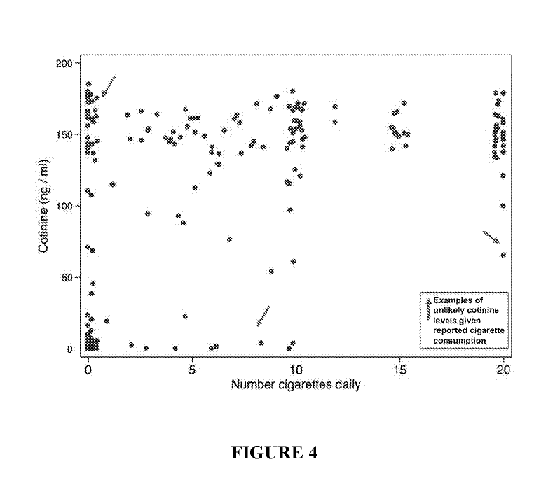

However, the relationship between COT levels and self-reported daily cigarette consumption is complex (FIG. 4). The actual distribution includes outliers of both types (high COT levels when reporting low or no cigarette-smoking; and low COT levels after reporting even high levels of smoking). As a result, a COT threshold of <100 ng/ml, e.g., is fairly successful in separating individuals who report not-smoking, with a true negative rate of 92.9%. However, the true positive rate is only 79.1%. The consequent “false positive” rate of nearly 21% must reflect, in addition to possible under-reporting, individual variation in nicotine metabolism, in smoking patterns (e.g., amount of nicotine in preferred brand, depth of inhalation, etc.), as well as other possible effects due to, e.g., age and gender.

### Example 6—Cg05575921 Methylation Levels and Reported Cigarette Consumption

As with COT, cg05575921 methylation levels are very different in smokers (median CpG: 70.9%, IQR: 63.3%-79.4%) compared to non-smokers (median CpG: 91.1%, IQR: 83.8%-94.9%; p<0.0001, Wilcoxon test). Using CpG levels alone, the optimum classifier reaches a sensitivity of 69.3% and a specificity of 95%, which results in a positive predictive value (PPV) of 86.3% and a negative predictive value (NPV) of 86.5%. Overall, 86% of individuals are correctly classified using CpG levels alone, and the AUC=0.89 (95% CI 0.86-0.92). These values are lower but relatively close to those obtained using the cotinine levels alone—except for sensitivity, which is lower for CpG (69%) than for COT (86%).

### Example 7—Combining COT Levels with Cg05575921 Methylation

Several observations suggest that CpG-levels are capturing information about smoking status above what is captured by COT. The two measures are strongly correlated (−0.55, p<0.0001, Spearman), but the correlation is insufficiently high to justify CpG levels as a proxy for COT. This is seen by focusing on the sub-samples where COT is high (>100 N=138) and a strong indicator of smoking, and where it is low (<50 ng/ml, N=252), indicative of non-smoking status. These thresholds are well within the range that the Benowitz et al. study would suggest are highly predictive of smoking or non-smoking status, respectively. Focusing now on the subset of the sample where COT >100, a very strong association was found between CpG methylation and reported smoking status (logistic regression, beta=−10.94, S.E. beta=2.29, p<0.0001, pseudo R2=22%). At the other extreme (low COT values <50; N=252), an association was also found between CpG methylation and reported smoking status (logistic regression, beta=−12.9, S.E. beta=4.6, p=0.005, pseudo R2=10%). This clearly demonstrates that methylation levels provide information above that provided by COT levels alone and the potential benefits of considering both in determining true smoking status.

### Example 8—Outline of the Algorithm

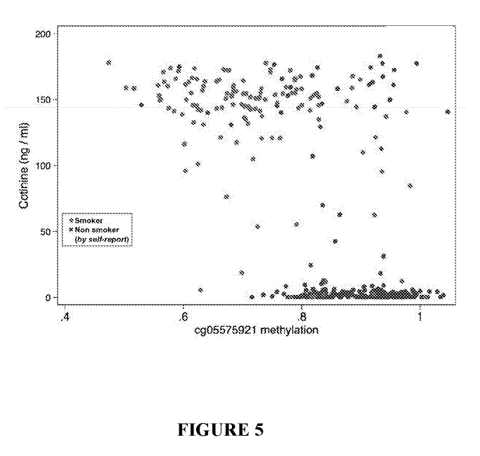

A two-step approach has been developed to leverage the information from the joint use of cotinine levels and cg05575921 methylation levels to predict smoking status. The approach uses established, albeit under-utilized, statistical methods. Indeed, a simple scatter plot of COT versus CpG (FIG. 5) readily shows that the usual algorithms (e.g., logistic based classification) are unlikely to succeed—and they do not. While definite trends are seen in the positioning of smokers and non-smokers (red and blue, respectively, by self-report), as confirmed by the logistic regressions summarized above, it is clear that even if the data is split by COT levels first, standard classification algorithms are unlikely to improve classification statistics (PPV, NPV, etc.) significantly above the use of one or the other measure alone.

Instead, in a first step, a non-parametric statistical approach was used (LOWESS; Cleveland, 1981, Am. Statist., 35:54) to predict COT levels as a function of age at assessment, gender, BMI, maximum of daily cigarettes in the previous 4 years and cg05575921 methylation levels. Second, both predicted COT levels and actual COT levels were use in developing a classifier to predict smoking status. This approach is distinguished from much work in this area, in that the approach described herein is actually leveraging the information from outliers. LOWESS is well established, but it is typically underutilized (compared, for example, to simple logistic regression) because it does not result in simple functional forms. Note further that the additional predictors can be collected at virtually no cost (e.g., self-reports from patient). As is shown below, the inclusion of cg05575921 methylation levels in the model is critical.

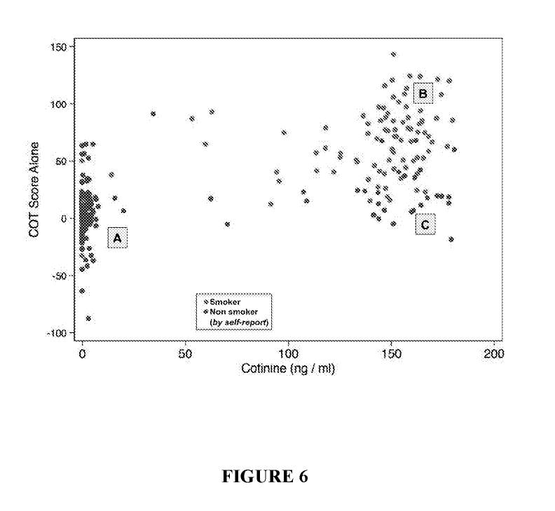

As a first indicator of the further information gained from CpG levels, consider FIG. 6, where only COT levels are used. The horizontal axis shows cotinine levels and the vertical axis shows COT score, i.e., the predicted cotinine levels given self-reported smoking history, gender and age. The difficulty is not so much with group (A), which is characterized by low COT and is composed primarily of non-smokers. Rather, it is with the lack of separation between groups (B, smokers) and (C, self-report non-smokers with unexpectedly high cotinine values).

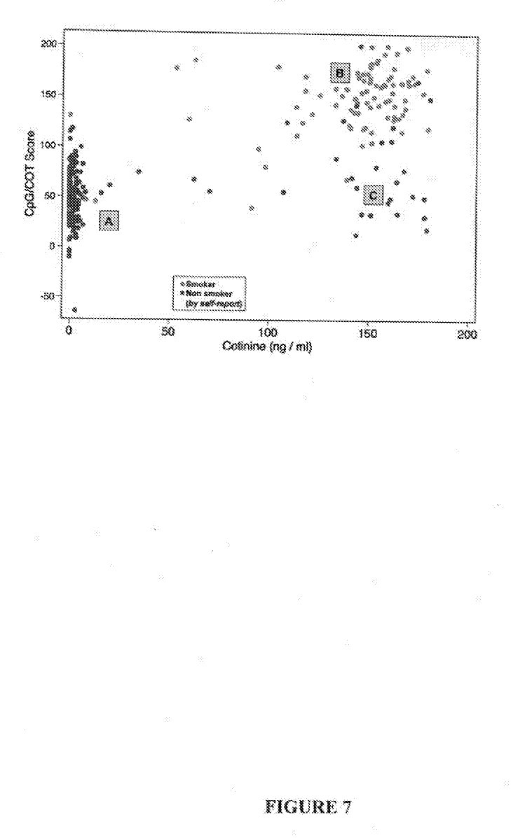

The separation between these two groups is greatly enhanced when cg05575921 methylation levels are entered into the model (FIG. 7). As in FIG. 6, where CpG methylation was not taken into account, the horizontal axis shows cotinine levels. In FIG. 7, the vertical axis now shows a combined COT/CpG score, i.e., the predicted cotinine levels given self-reported smoking history, gender, age, and cg05575921 methylation level. The separation between the two difficult groups (B and C) is greatly enhanced.

### Example 9—Cluster Analyses Reveals Further Benefits Over the Joint COT/CpG Score

Compared to using COT levels alone, the combined approach described herein raises the sensitivity to 91% (up from 86%) and the specificity to 96% (up from 89%), which results in a positive predictive value to 90% (up from 79%) and a negative predictive value to 96% (up from 93%). Overall, 95% of individuals are correctly classified (up from 88%) and the AUC is increased to 0.96 (up from 092). While these improvements may seem small, a cluster analysis highlights the true benefit.

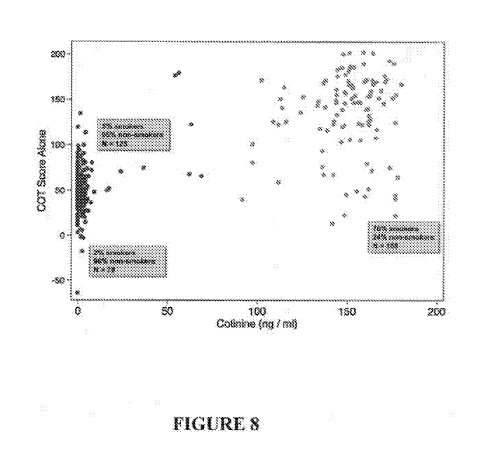

FIG. 8, which is based on cotinine scores alone, adjusting for gender, age and smoking history summarizes the results of cluster analysis on predicted COT score and observed cotinine levels (k-means clustering). It can be seen that using COT alone, as has been alluded to above, two relatively clean clusters of non-smokers are identified (green, blue) but, with cotinine levels alone, it is difficult to distinguish between smokers and non-smokers for a large portion of the subjects (108 subjects assigned, with 24% contamination).

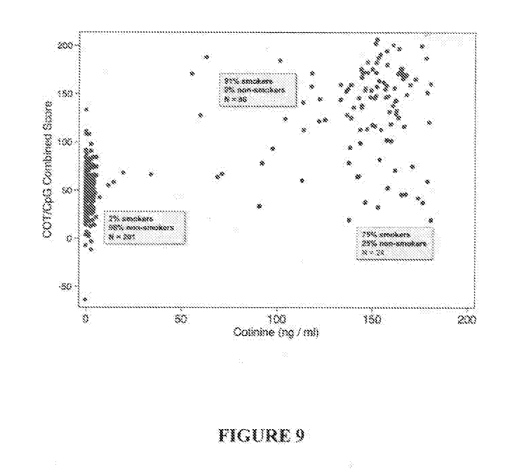

However, when cg05575921 methylation levels are taken into account (FIG. 9), the same clustering technique reveals a clean cluster of non-smokers (N=201, 2% contamination), a clean cluster of smokers (N=86, 9% contamination) and a far smaller cluster of uncertain cases (N=24, 25% contamination, blue).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of determining whether or not an individual is a tobacco user, comprising the steps of:
determining the level of cotinine in a biological sample from the individual;
determining the methylation status of at least one CpG dinucleotide in a biological sample from the individual; and
correlating the level of cotinine and the methylation status in the biological sample to determine whether or not the individual is a tobacco user.

2. The method of claim 1, wherein the level of cotinine is determined using ELISA.

3. The method of claim 1, wherein the methylation status of the at least one CpG dinucleotide is determined using bi-sulfite treated DNA.

4. The method of claim 1, wherein the correlating step comprises applying an algorithm.

5. The method of claim 1, wherein the biological sample is selected from the group consisting of peripheral blood, lymphocytes, urine, saliva, and buccal cells.

6. The method of claim 1, wherein the at least one CpG dinucleotide comprises position 373378 of chromosome 5 in the AHRR gene.

7. The method of claim 6, wherein demethylation at position 373378 of chromosome 5 is indicative of previous or current tobacco use.

8. The method of claim 1, wherein the at least one CpG dinucleotide comprises position 377358 of chromosome 5 in the AHRR gene or position 399360 of chromosome 5 in the AHRR gene.

9. The method of claim 8, wherein demethylation at position 377358 of chromosome 5 or at position 399360 of chromosome 5 is indicative of previous or current tobacco use.

10. The method of claim 1, further comprising obtaining self-report data from the individual regarding whether or not the individual is a tobacco user.

11. A computer implemented method for determining whether or not an individual is a tobacco user, the method comprising:
obtaining, at a computer system, information regarding at least one event that is associated with a user;
performing one or more predictive calculations for the user, the calculations based, at least in part, on the obtained information;
obtaining measured data associated with the user, the measured data comprising one or more measured COT levels and one or more measured CpG methylation status;
generating a predictive score based on the obtained information, the predictive calculations, and the measured data; and
providing a likelihood of tobacco usage by the user based on the predictive score.

12. The method of claim 11, wherein the information comprises at least one of age, gender, race, ethnicity, tobacco use, and genotype.

13. The method of claim 11, wherein the one or more predictive calculations comprises a predicted COT level and/or a predicted CpG methylation status.

14. The method of claim 13, wherein the generating a predictive score comprises obtaining a bivariate score between predicted COT levels and predicted CpG methylation status and measured COT levels and measured CpG methylation status.

15. The method of claim 11, further comprising:
generating the score using the information and the CpG methylation status when the predicted COT level for the user and/or the measured COT level for the user is below a threshold.

16. The method of claim 11, further comprising:
determining the CpG methylation status for the user, wherein a change in methylation status is an indicator of tobacco use.

17. A computer implemented method for determining whether or not an individual is a tobacco user, the method comprising:
obtaining self-report data for a user;
performing one or more predictive calculations to determine a predicted COT level, a predicted CpG methylation status and predicted tobacco use of the user;
providing a measured COT level and a measured CpG methylation status for the user;
generating a predictive score based on the self-report data, the one or more predictive calculations, the measured COT level and the measured CpG methylation status; and
outputting a predicted level of tobacco usage based on the predictive score.

18. A decision support system comprising:
a processor;
a storage device coupled to the processor and storing instructions that, when executed by the processor, cause the processor to perform operations comprising correlating COT levels in an individual and methylation status in the individual with tobacco use by the individual.
